(19) United States
(12) Patent Application Publication
(10) Pub. No.: US 2021/0120810 A1
(43) Pub. Date: Apr. 29, 2021

(54) INSECT PATHOGENIC MICROORGANISM-CONTAINING AGRICULTURAL PESTICIDE FORMULATION AND MANUFACTURING METHOD THEREFOR

(71) Applicant: Kyung Bon KOO, Daegu (KR)

(72) Inventors: Kyung Bon KOO, Daegu (KR); Hyung Chul LEE, Daegu (KR)

(21) Appl. No.: 17/051,039

(22) PCT Filed: Apr. 29, 2019
(86) PCT No.: PCT/KR2019/005139

§ 371 Date: Oct. 27, 2020

(30) Foreign Application Priority Data
Apr. 30, 2018 (KR) 10-2018-0049556

(51) Int. Cl. A01N 25/12; A01N 25/08; A01N 63/30

(52) U.S. Cl. CPC A01N 25/12; A01N 63/30; A01N 25/08

(57) ABSTRACT

The present disclosure relates to a pesticide formulation containing an insect-pathogenic microorganism and a method of manufacturing the same, and more particularly to a granular or powder pesticide formulation containing an insect-pathogenic microorganism and a method of manufacturing the same. The granular pesticide formulation of the present disclosure includes (a) a porous carrier, (b) a medium infiltrated into pores in the porous carrier, and (c) an insect-pathogenic microorganism cultured in the medium. The pesticide formulation containing the insect-pathogenic microorganism according to the present disclosure has a superior insecticidal effect because the number of spores of the insect-pathogenic microorganism having insecticidal activity is significantly increased compared to existing formulations.

### TECHNICAL FIELD

The present disclosure relates to a pesticide formulation containing an insect-pathogenic microorganism and a method of manufacturing the same, and more particularly to a granular or powder pesticide formulation containing an insect-pathogenic microorganism and a method of manufacturing the same.

### BACKGROUND ART

Plant diseases caused by pathogens such as insects impose a high economic burden on agricultural and plant-based industries. Damage occurring both before and after harvest of plants, loss of the plant itself, or loss due to reduced growth capacity and productivity may be significant.

Traditionally, control of plant pathogens has been achieved using a number of chemical pesticides. The use of chemicals has many drawbacks. Pathogens may develop resistance to chemicals over time, and they create resistant populations due to the resistance thus obtained. Indeed, resistance to pesticides is the greatest challenge to the viability of the horticultural industry.

Pesticides for controlling crop pests are known to cause side effects such as resistance thereto and environmental pollution during long-term or excessive use. In order to solve this problem, pest control using microorganisms has been proposed as an alternative thereto, and there are 171 types of fungal pesticides registered for pest control worldwide, which are widely used for control of pests such as moths, Coleoptera, aphids, powdery mildew and the like. Three types of fungal insecticides are registered in Korea. When spraying insect-pathogenic fungi on crops for pest control, high concentrations of spores are generally applied. Therefore, mass production of insecticidal fungal spores is a critical goal that must be achieved first in developing fungal insecticides. In general, three methods (solid culture, liquid culture, and two-phase fermentation of liquid-solid culture) are used for mass culture of insect-pathogenic fungal spores.

Although insect-pathogenic fungi are capable of producing spores through liquid culture, spores resulting from liquid culture are hydrophilic, so there is a problem in that they are not easily included in oil formulations.

Solid culture using grains such as rice, barley, corn, wheat bran, etc. as a medium is advantageous because of the high viability of mold and ease of sporulation, but there is a problem of reduced production efficiency and increased production and distribution costs because unnecessary insoluble medium components that are not used as nutrients in the medium are included in all processing steps. Due to the large amount of insoluble substances, there may occur dispersion stability problems upon hydration, problems upon passing through a spray nozzle, and the like.

Korean Patent No. 1800330 discloses a liquid medium composition for improving sporulation of Beauveria bassiana containing glucose, a yeast extract, MgSO₄.7H₂O and K₂HPO₄, and Korean Patent No. 1499692 discloses a method of producing fungal spores in which insect-pathogenic fungi are cultured in a solid medium for a natural zeolite ceramic ball carrier.

However, it is necessary to develop a pesticide formulation that is capable of further increasing sporulation efficiency and being easily processed.

Therefore, the inventors of the present disclosure have endeavored to solve the above problems, and have ascertained that, when a porous carrier in which a medium is infiltrated into the pores thereof is inoculated with an insect-pathogenic microorganism and is cultured, a granular pesticide formulation in which the number of spores of the insect-pathogenic microorganism having insecticidal activity is increased may be manufactured, thus culminating in the present disclosure.

### DISCLOSURE

### Technical Problem

An objective of the present disclosure is to provide a pesticide formulation containing a large amount of insect-pathogenic microorganism spores and a method of manufacturing the same.

### Technical Solution

In order to accomplish the above objective, the present disclosure provides a granular pesticide formulation, including: (a) a porous carrier; (b) a medium infiltrated into pores in the porous carrier; and (c) an insect-pathogenic microorganism cultured in the medium.

In addition, the present disclosure provides a method of manufacturing a granular pesticide formulation, including: (a) allowing a medium for culturing an insect-pathogenic microorganism to infiltrate into pores in a porous carrier; and (b) inoculating the porous carrier, into which the medium is infiltrated, with an insect-pathogenic microorganism and performing culture.

In the present disclosure, the porous carrier may be a porous ceramic ball or sponge.

In the present disclosure, the porous ceramic ball may be selected from the group consisting of a zeolite ball, a terra alba ball, a loess ball, a magnetite ball, and a diatomite ball.

In the present disclosure, the sponge may be foam rubber or a urethane-based resin.

In the present disclosure, the medium may be selected from the group consisting of whole milk powder, skim milk powder, whey, milk, wheat flour, starch, and grain powder.

In the present disclosure, the insect-pathogenic microorganism may be a fungus that has insecticidal activity against pests and forms spores.

In addition, the present disclosure provides a powder pesticide formulation, including: (a) an insect-pathogenic microorganism; (b) cellulose; and (c) diatomite or zeolite.

In the present disclosure, the powder pesticide formulation may include 15 to 25 wt % of the insect-pathogenic microorganism, 20 to 40 wt % of the cellulose, and 40 to 60 wt % of the diatomite or zeolite.

### Advantageous Effects

According to the present disclosure, a pesticide formulation containing an insect-pathogenic microorganism has a superior insecticidal effect because the number of spores of the insect-pathogenic microorganism having insecticidal activity is significantly increased compared to existing formulations.

### BRIEF DESCRIPTION OF DRAWINGS

FIG. 1 shows photographs of granular pesticide formulations manufactured according to an embodiment of the present disclosure;

FIG. 2 shows photographs of the granular pesticide formulations manufactured according to an embodiment of the present disclosure, depending on the amount of a medium;

FIG. 3 shows changes over time in a spore germination rate of the granular pesticide formulations manufactured according to an embodiment of the present disclosure and micrographs of the spores thereof;

FIG. 4 shows photographs of the granular pesticide formulations manufactured according to an embodiment of the present disclosure, depending on the type of a porous carrier;

FIG. 5 shows photographs of the granular pesticide formulations manufactured according to an embodiment of the present disclosure, depending on the type of porous carrier and the type of insect-pathogenic microorganism;

FIG. 6 shows photographs of powder pesticide formulations manufactured according to an embodiment of the present disclosure;

FIG. 7 shows changes over time in a spore germination rate of the powder pesticide formulations manufactured according to an embodiment of the present disclosure and micrographs of the spores thereof;

FIG. 8 shows photographs of powder pesticide formulations manufactured according to an embodiment of the present disclosure; and

FIG. 9 shows changes over time in a spore germination rate of the powder pesticide formulations manufactured according to an embodiment of the present disclosure and micrographs of the spores thereof.

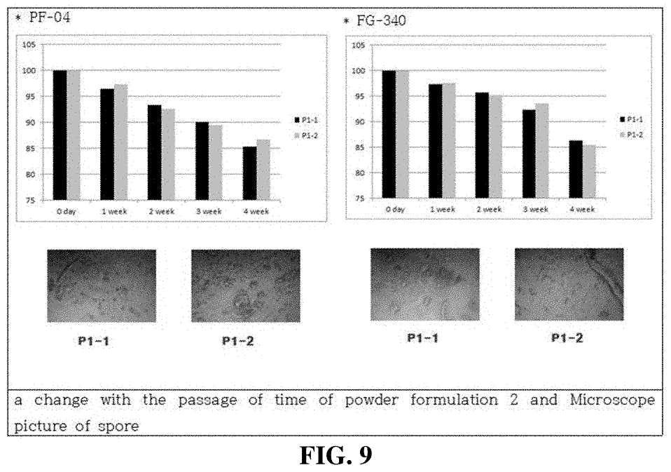

### BEST MODE

The present disclosure is intended to confirm that, when a porous carrier in which a medium is infiltrated into the pores thereof is inoculated with an insect-pathogenic microorganism and is cultured, a granular pesticide formulation having an increased number of spores of the insect-pathogenic microorganism having insecticidal activity may be manufactured.

In the present disclosure, a granular pesticide formulation is manufactured in a manner in which whole milk powder is allowed to infiltrate into pores in a porous ceramic ball or sponge serving as a porous carrier, followed by inoculation with an insect-pathogenic microorganism, for example, Isaria javanica Pf04 or Isaria fumosorosea FG340, and then culture. Thereby, it can be confirmed that the manufactured granular pesticide formulation has a significantly increased number of spores of the insect-pathogenic microorganism, compared to when using Panicum miliaceum as a typical solid culture medium.

An aspect of the present disclosure pertains to a granular pesticide formulation, including (a) a porous carrier, (b) a medium infiltrated into pores in the porous carrier, and (c) an insect-pathogenic microorganism cultured in the medium.

The porous carrier serves to facilitate culture and sporulation of the insect-pathogenic microorganism, and it is possible to use those having therein pores into which the medium for culturing an insect-pathogenic microorganism is capable of infiltrating. Examples of the porous carrier may include, but are not limited to, a porous ceramic ball, sponge, and the like.

Examples of the porous ceramic ball may include a zeolite ball, a terra alba ball, a loess ball, a magnetite ball, a diatomite ball, etc., and the diameter of the porous ceramic ball is not particularly limited, but is preferably 0.5 to 30 mm. If the diameter of the ceramic ball is less than 0.5 mm, it is difficult to perform infiltration of the medium into the pores and inoculation with the insect-pathogenic microorganism due to the small size thereof. On the other hand, if the diameter thereof exceeds 30 mm, the use thereof may be inefficient due to the large size thereof.

Also, various kinds of sponge may be used regardless of the type of material, so long as it is able to achieve the above purpose, and examples thereof may include foam rubber, a urethane-based resin, and the like.

In the present disclosure, the medium is used for culture and sporulation of the insect-pathogenic microorganism, and may vary depending on the type of insect-pathogenic microorganism, and examples thereof include whole milk powder, skim milk powder, whey, milk, wheat flour, starch and grain powder, with the use of whole milk powder being preferable.

In the present disclosure, the insect-pathogenic microorganism may be used without limitation, so long as it has insecticidal activity against pests, and the use of a spore-forming fungus is preferable.

The granular pesticide formulation may be manufactured by inoculating a porous carrier in which a medium is infiltrated into the pores thereof with an insect-pathogenic microorganism and performing culture.

Another aspect of the present disclosure pertains to a method of manufacturing a granular pesticide formulation, including (a) allowing a medium for culturing an insect-pathogenic microorganism to infiltrate into pores in a porous carrier, and (b) inoculating the porous carrier, into which the medium is infiltrated, with an insect-pathogenic microorganism and performing culture.

In the granular pesticide formulation of the present disclosure, the medium is allowed to infiltrate into the pores in the porous carrier, after which the porous carrier is inoculated with the insect-pathogenic microorganism and cultured, whereby a higher number of spores may be formed, rather than spraying an insect-pathogenic microorganism culture solution onto the surface.

The number of insect-pathogenic microorganisms inoculated into the porous carrier into which the medium is infiltrated is not particularly limited, but is preferably 1-10×10⁸ cfu/g or more.

The insect-pathogenic microorganism inoculated into the porous carrier into which the medium is infiltrated may be cultured under various conditions depending on the type of insect-pathogenic microorganism, but culture is preferably performed at 25 to 30° C. for 4 to 7 days.

In the present disclosure, the granular pesticide formulation that is ultimately manufactured contains spores in an amount of 1×10⁹ cfu/g or more.

The present disclosure is intended to confirm that, when cellulose and ceramic are used along with the insect-pathogenic microorganism, a powder pesticide formulation having an increased sporulation rate may be manufactured without agglomeration.

In the present disclosure, a powder pesticide formulation, containing Isaria javanica Pf04 or Isaria fumosorosea FG340, which is the insect-pathogenic microorganism, cellulose, and each of vermiculite, diatomite, and zeolite, is manufactured. Thereby, it can be confirmed that the powder pesticide formulation, manufactured using cellulose and diatomite or cellulose and zeolite along with the insect-pathogenic microorganism, has a significantly increased number of spores of the insect-pathogenic microorganism compared to when using Panicum miliaceum as a typical solid culture medium.

Still another aspect of the present disclosure pertains to a powder pesticide formulation, including (a) an insect-pathogenic microorganism, (b) cellulose, and (c) diatomite or zeolite.

The insect-pathogenic microorganism may be used without limitation, so long as it has insecticidal activity against pests, and the use of a spore-forming fungus is preferable. The cellulose may be used alone in a powder pesticide formulation, but is problematic in that it is expensive and agglomerates over time. Therefore, cellulose is preferably used along with ceramic, and examples of the ceramic include diatomite, zeolite, and the like.

The powder pesticide formulation may contain 15 to 25 wt % of the insect-pathogenic microorganism, 20 to 40 wt % of cellulose, and 40 to 60 wt % of diatomite or zeolite. When the amounts of the insect-pathogenic microorganism, cellulose, and diatomite or zeolite fall out of the above ranges, there may be a problem in that sporulation does not occur properly.

### Mode for Disclosure

A better understanding of the present disclosure will be given through the following examples. These examples are merely set forth to illustrate the present disclosure, and are not to be construed as limiting the scope of the present disclosure, as will be apparent to those skilled in the art.

### EXAMPLE 1: PREPARATION OF GRANULAR PESTICIDE FORMULATION

1-1: Investigation of Optimal Medium Composition for Manufacturing Granular Pesticide

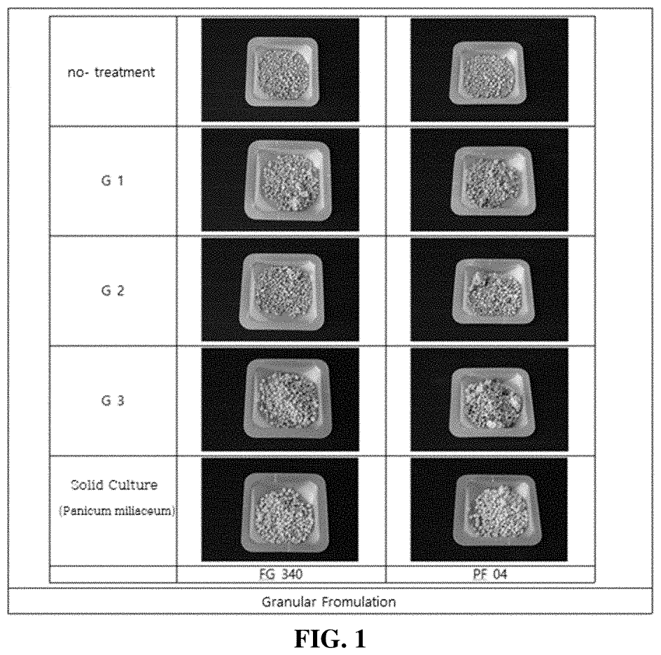

As shown in Table 1 below, each of potato starch, corn starch, and whole milk powder was mixed with water, after which 20 ml of the medium (mixed solution) was uniformly mixed in 80 g of zeolite balls (diameter: 1-2 mm) and was allowed to infiltrate into the pores thereof. The porous carrier into which the medium was infiltrated was sterilized and was then inoculated with 1 ml (1-10×10⁸ cfu/ml) of each of Isaria javanica Pf04 (KACC93122P) and Isaria fumosorosea FG340 (KACC93199P), serving as an insect-pathogenic microorganism, relative to 100 g of zeolite balls, and cultured at 25° C. for 5 days (FIG. 1). Here, Panicum miliaceum was used as a control.

The number of insect-pathogenic microorganism spores that were cultured was measured using a hemocytometer, and the results thereof are shown in Table 2 below.

TABLE 1

| Component | No treatment | G1 | G2 | G3 |
|---|---|---|---|---|
| Zeolite | 100% | 80% | 80% | 80% |
| Potato starch | | 4% | | |
| Corn starch | | | 4% | |
| Whole milk powder | | | | 4% |
| Water | | 16% | 16% | 16% |
| Total (wt %) | 100% | 100% | 100% | 100% |

TABLE 2

| | Isaria fumosorosea FG340 | Isaria javanica Pf04 |
|---|---|---|
| No treatment | — | — |
| G1 | 8 × 10⁷ | 8 × 10⁷ |
| G2 | 1.6 × 10⁸ | 8 × 10⁷ |
| G3 | 1.04 × 10⁹ | 7.2 × 10⁸ |
| Solid culture | 9.6 × 10⁸ | 1.26 × 10⁹ |
| (Panicum miliaceum) | | |

As is apparent from Table 2, in the case of Isaria fumosorosea FG340, the number of spores was high in the order of G3>solid culture (Panicum miliaceum)>G2>G1, and in the case of Isaria javanica Pf04, the number of spores was high in the order of solid culture>G3>G2=G1.

Therefore, it was found that whole milk powder was most suitable as the granular solid culture medium using zeolite balls.

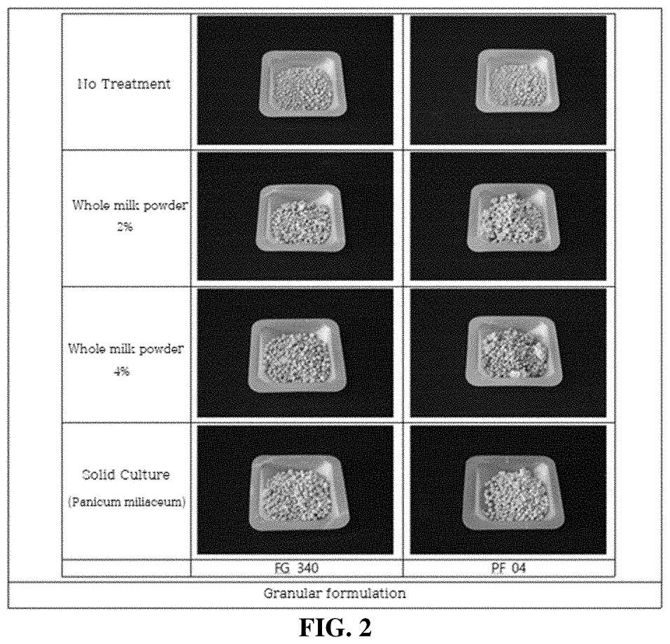

In order to confirm the optimal ratio when using whole milk powder as the medium, a whole milk powder medium was prepared using components in the amounts shown in Table 3 below, and was then allowed to infiltrate into the pores in zeolite balls in the same manner, followed by inoculation with an insect-pathogenic microorganism and culture (FIG. 2). The number of insect-pathogenic microorganism spores that were obtained was measured using a hemocytometer, and the results thereof are shown in Table 4 below.

TABLE 3

| Component | No treatment | G3-1 | G3-2 |
|---|---|---|---|
| Zeolite | 100% | 80% | 80% |
| Whole milk powder | | 2% | 4% |
| Water | | 18% | 16% |
| Total (wt %) | 100% | 100% | 100% |

TABLE 4

| | Isaria fumosorosea FG340 | Isaria javanica Pf04 |
|---|---|---|
| No treatment | — | — |
| G3-1 | 1.12 × 10⁹ | 1.2 × 10⁹ |
| G3-2 | 1.04 × 10⁹ | 7.2 × 10⁸ |
| Solid culture | 9.6 × 10⁸ | 1.26 × 10⁹ |
| (Panicum miliaceum) | | |

As is apparent from Table 4, in the case of Isaria fumosorosea FG340, the growth of spores was similar depending on the proportion of whole milk powder, but in the case of Isaria javanica Pf04, it was confirmed that the number of spores was higher when the proportion of whole milk powder was low (2%) than when the proportion of whole milk powder was high (4%).

1-2: Investigation of Porous Carrier for Manufacturing Granular Pesticide

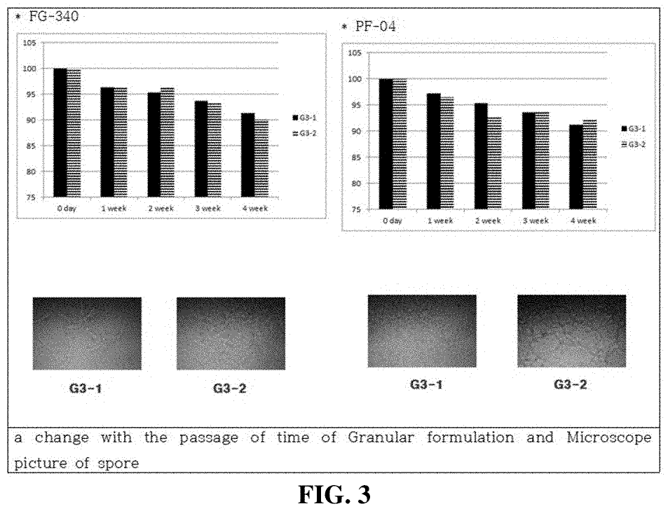

2 wt % of whole milk powder and 18 wt % of water were mixed, after which the medium was allowed to infiltrate into the pores in the porous carrier. The porous carrier in which the medium was infiltrated into the pores thereof (zeolite balls: 0.5 to 2 mm, terra alba balls: 3 mm, loess balls: 5 mm, magnetite balls: 6 mm, diatomite balls: 10 mm, terra alba balls: 25 mm, sponge (polyurethane, Goldstar sponge) 10×10×20 mm) was sterilized, inoculated with 1 ml (1-10×10⁸ cfu/ml) of each of Isaria javanica Pf04 (KACC93122P) and Isaria fumosorosea FG340 (KACC93199P), serving as an insect-pathogenic microorganism, relative to 100 g of the porous carrier, and cultured at 25° C. for 5 days (FIG. 3). The porous carrier was uniformly mixed every 12 hr during culture.

As shown in FIG. 3, the spores of the Isaria javanica Pf04 strain and the Isaria fumosorosea FG340 strain were well formed on the surface of all of the zeolite balls, terra alba balls, loess balls, magnetite balls, diatomite balls and sponge.

1-3: Investigation of Insect-Pathogenic Microorganism for Manufacturing Granular Pesticide

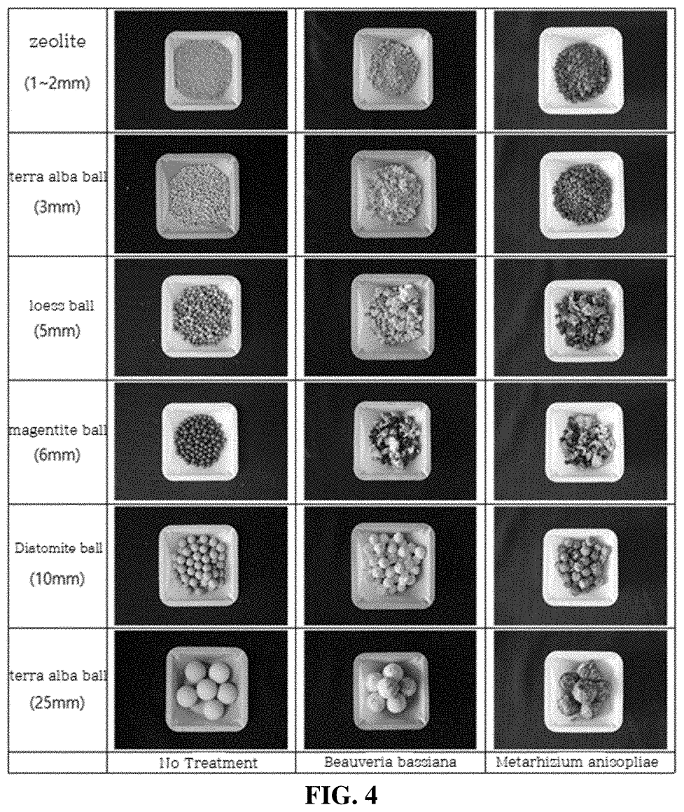

The porous carrier into which the medium was infiltrated was inoculated with an insect-pathogenic microorganism and was cultured in the same manner as in Example 1-2, with the exception that Beauveria bassiana (KACC 40377) and Metarhizium anisopliae (KACC 40969) were used as the insect-pathogenic microorganism, in lieu of Isaria javanica Pf04 (KACC93122P) and Isaria fumosorosea FG340 (KACC93199P) (FIG. 4).

As shown in FIG. 4, the spores of Beauveria bassiana (KACC 40377) and Metarhizium anisopliae (KACC 40969) were well formed on the surface of all of the zeolite balls, loess balls, magnetite balls, diatomite balls, and terra alba balls.

Specifically, in the spraying process, it is difficult to uniformly spray the spray solution on the ceramic balls because the carrier contains no medium component, and uniform growth thereof is also difficult. However, when the medium component is directly allowed to infiltrate into the carrier (ceramic balls), even if the inoculation is not performed evenly, bacteria are capable of growing and spreading efficiently by hyphae or spores, thus enabling uniform growth thereof.

### EXAMPLE 2: PREPARATION OF POWDER PESTICIDE FORMULATION

2-1: Investigation of Optimal Medium Composition for Manufacturing Powder Pesticide

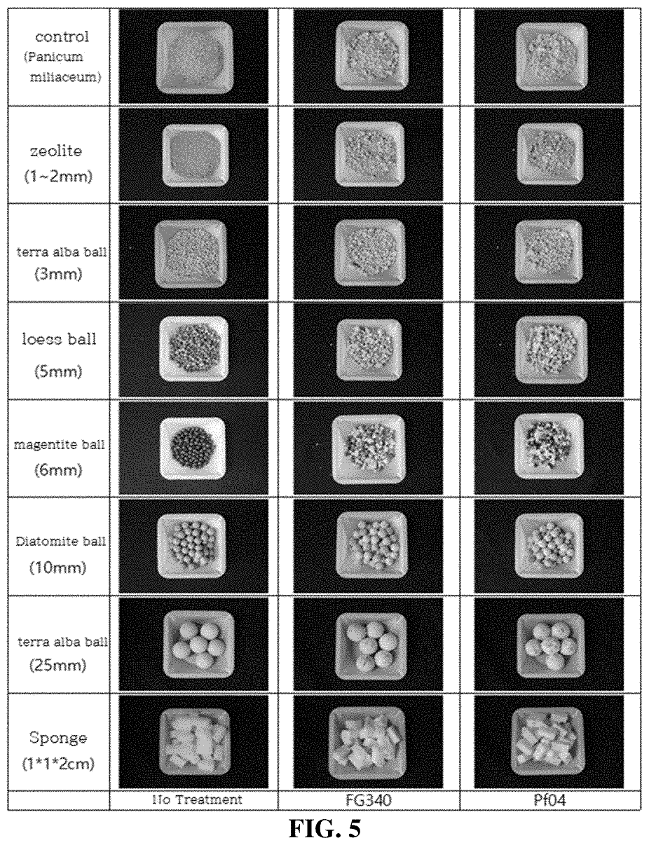

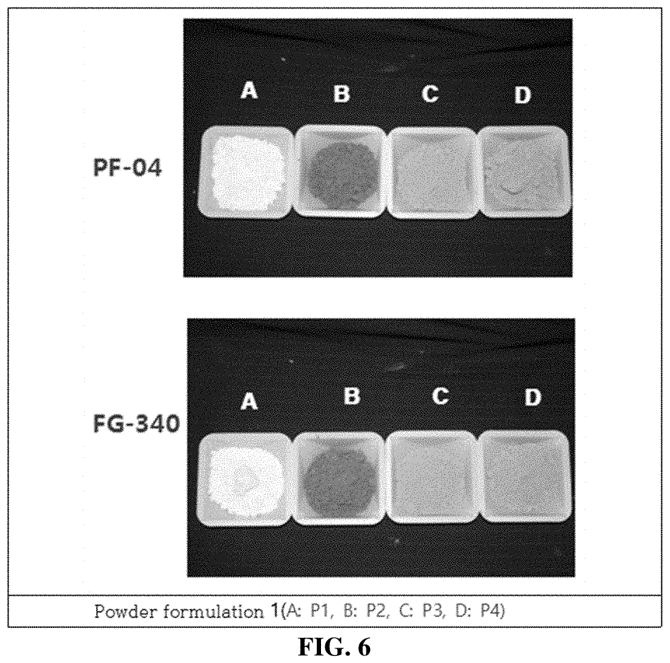

As shown in Table 5 below, a powder pesticide formulation was manufacturing by mixing an insect-pathogenic microorganism culture solution with each of cellulose, vermiculite, diatomite, and zeolite (FIG. 5), and then a viable cell count was taken and spore germination was observed for 4 weeks at 54° C., and the results thereof are shown in FIG. 6.

Here, the insect-pathogenic microorganism that was used was one (1-10×10⁸ cfu/ml) obtained by inoculating an EC 1 medium (based on 1 l of water, 10 g of whole milk powder, 15 g of peptone, 2.5 g of egg yolk powder, 1 g of yeast, 5 g of sodium chloride, 2.5 g of potassium monophosphate) with each of Isaria javanica Pf04 (KACC93122P) and Isaria fumosorosea FG340 (KACC93199P), followed by culture at 28° C. at 150 rpm for 5 days.

TABLE 5

| Component | P1 | P2 | P3 | P4 |
|---|---|---|---|---|
| Culture solution | 20% | 20% | 20% | 20% |
| Cellulose | 80% | | | |
| Vermiculite | | 80% | | |
| Diatomite | | | 80% | |
| Zeolite | | | | 80% |
| Total (wt %) | 100% | 100% | 100% | 100% |

As shown in FIG. 6, in both Isaria fumosorosea FG340 and Isaria javanica Pf04, it was confirmed that the bacterial density was maintained in the order of P1>P3=P4>P2. Briefly, the germination rate was maintained even when the storage time elapsed.

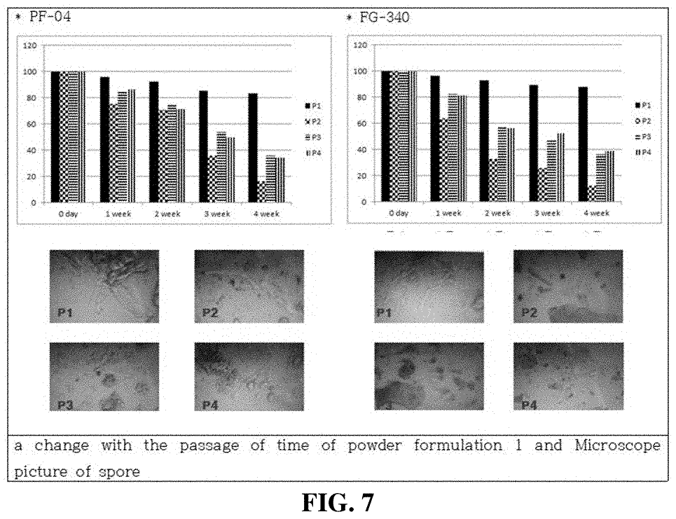

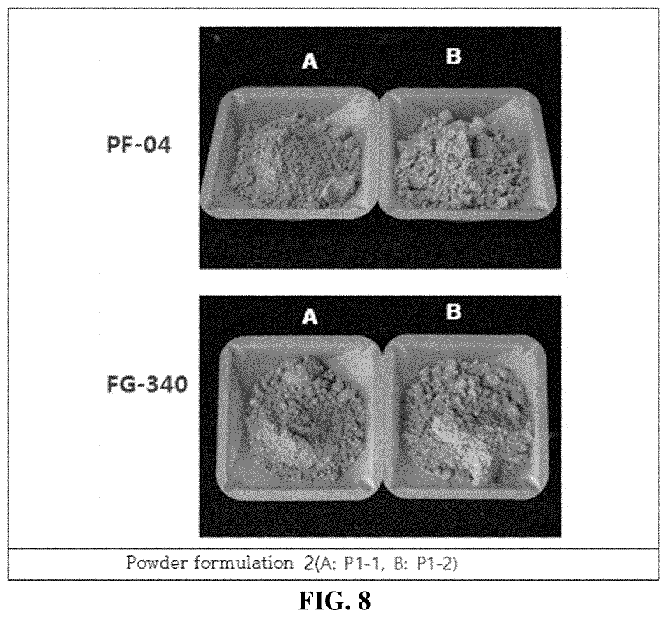

Therefore, it was found that the P1 formulation is most suitable. However, since cellulose, the material of P1, is problematic in that the unit price thereof is high and the formulation agglomerates over time, cellulose was mixed with different types of powder as shown in Table 6 below to afford a powder pesticide formulation (FIG. 7), after which a viable cell count was taken and spore germination was observed for 4 weeks at 54° C., and the results thereof are shown in FIG. 8.

TABLE 6

| Component | P1-1 | P1-2 |
|---|---|---|
| Culture solution | 20% | 20% |
| Cellulose | 30% | 30% |
| Vermiculite | | |
| Diatomite | 50% | |
| Zeolite | | 50% |
| Total (wt %) | 100% | 100% |

As shown in FIG. 8, when cellulose and diatomite or cellulose and zeolite were used, both Isaria fumosorosea FG340 and Isaria javanica Pf04 exhibited a germination rate of 85% or more on the 4^{th} week without agglomeration, indicative of superior effects.

Although specific embodiments of the present disclosure have been disclosed in detail above, it will be obvious to those skilled in the art that the description is merely of preferable exemplary embodiments and is not to be construed to limit the scope of the present disclosure. Therefore, the substantial scope of the present disclosure will be defined by the appended claims and equivalents thereof.

### INDUSTRIAL APPLICABILITY

According to the present disclosure, a pesticide formulation containing an insect-pathogenic microorganism has a superior insecticidal effect and is thus industrially useful because the number of spores of the insect-pathogenic microorganism having insecticidal activity is significantly increased compared to existing formulations.

1. A granular pesticide formulation, comprising:
(a) a porous carrier;
(b) a medium infiltrated into pores in the porous carrier; and
(c) an insect-pathogenic microorganism cultured in the medium.

2. The granular pesticide formulation of claim 1, wherein the porous carrier is a porous ceramic ball or sponge.

3. The granular pesticide formulation of claim 2, wherein the porous ceramic ball is selected from the group consisting of a zeolite ball, a terra alba ball, a loess ball, a magnetite ball, and a diatomite ball.

4. The granular pesticide formulation of claim 2, wherein the sponge is foam rubber or a urethane-based resin.

5. The granular pesticide formulation of claim 1, wherein the medium is selected from the group consisting of whole milk powder, skim milk powder, whey, milk, wheat flour, starch, and grain powder.

6. The granular pesticide formulation of claim 1, wherein the insect-pathogenic microorganism is a fungus that has insecticidal activity against pests and forms spores.

7. A method of manufacturing a granular pesticide formulation, comprising:
(a) allowing a medium for culturing an insect-pathogenic microorganism to infiltrate into pores in a porous carrier; and
(b) inoculating the porous carrier, into which the medium is infiltrated, with an insect-pathogenic microorganism and performing culture.

8. The method of claim 7, wherein the porous carrier is a porous ceramic ball or sponge.

9. The method of claim 8, wherein the porous ceramic ball is selected from the group consisting of a zeolite ball, a terra alba ball, a loess ball, a magnetite ball, and a diatomite ball.

10. The method of claim 8, wherein the sponge is foam rubber or a urethane-based resin.

11. The method of claim 7, wherein the medium is selected from the group consisting of whole milk powder, skim milk powder, whey, milk, wheat flour, starch, and grain powder.

12. The method of claim 7, wherein the insect-pathogenic microorganism is a fungus that has insecticidal activity against pests and forms spores.

13. A powder pesticide formulation, comprising:
(a) an insect-pathogenic microorganism;
(b) cellulose; and
(c) diatomite or zeolite.

14. The powder pesticide formulation of claim 13, wherein the powder pesticide formulation comprises 15 to 25 wt % of the insect-pathogenic microorganism; 20 to 40 wt % of the cellulose; and 40 to 60 wt % of the diatomite or zeolite.
